(19) United States
(12) Patent Application Publication
(10) Pub. No.: US 2007/0092490 A1
(43) Pub. Date: Apr. 26, 2007

(54) HIGH EFFICIENCY SIN VECTOR

(76) Inventors: Kwan Hee LEE, Gaithersburg, MD (US); Youngsuk Yi, Gaithersburg, MD (US)

Correspondence Address: JHK LAW, P.O. BOX 1078, LA CANADA, CA, 91012-1078, US

(21) Appl. No.: 11/551,659

(22) Filed: Oct. 20, 2006

Related U.S. Application Data
(60) Provisional application No. 60/596,788, filed on Oct. 20, 2005.

(51) Int. Cl. A61K 48/00; C12N 15/86; C12N 5/06

(52) U.S. Cl. 424/93.2; 435/456; 435/325

(57) ABSTRACT

The present application discloses viral vector that includes the following elements: (1) a promoter in U3 region of MSV 5′LTR; (2) repeating unit of MSV 5′LTR; (3) U5 region of MSV 5′LTR; (4) packaging signal; (5) a promoter; (6) internal ribosome entry site (IRES); (7) defective MLV 3′ LTR; (8) repeating unit of MLV 3′ LTR; and (9) U5 region of MLV 3′ LTR.

### CROSS-REFERENCE TO RELATED APPLICATIONS

The present application claims the benefit of priority to U.S. Provisional Patent Application No. 60/596,788, filed Oct. 20, 2005, the contents of which are incorporated by reference in their entirety, and U.S. patent application Ser. No. 11/160,066, filed Jun. 7, 2005, the contents of which are incorporated by reference in their entirety.

### BACKGROUND OF THE INVENTION

1. Field of the Invention

The present invention relates to the field of recombinant vectors. The present invention relates to the field of recombinant vectors as they are used in gene therapy.

2. General Background and State of the Art

Retroviral vectors have several advantages to be used as preferred gene transfer vectors in clinical gene therapy trials. These include their high efficiency of transduction into a variety of cell types and ability to integrate into the host cell chromosome allowing for a relatively stable expression of the incorporated genes (Palu, G. et al., Rev Med Virol. 2000 10 185-202; Hawley, R. G., Curr Gene Ther. 2001 1 1-17; Pfeifer, A. and Verma, I. M., Annu Rev Genomics Hum Genet. 2001 2 177-211; Robbins, P. D. et al., Trends Biotechnol. 1998 16 35-40). In the retroviral vectors currently used, the majority of the protein coding sequences for gag, pol and env genes are removed from the viral backbone making them deficient for viral replication. These three major viral proteins are provided in trans in the vector packaging system, either via co-transfecting plasmid constructs expressing genes for these proteins or from packaging cells in which these genes are pre-integrated into the genome (Danos, O. and Mulligan, R. C., Proc. Natl. Acad. Sci. U.S.A. 1988 85 6460-6464; Miller, A. D., Hum. Gene Ther. 1990 1 5-14). The remaining viral backbone contains minimum sequence necessary for encapsidation of the viral RNA (ψ packaging signal sequences), reverse transcription of the viral RNA and integration of proviral DNA (long terminal repeat regions, the transfer RNA-primer binding site, and a region including the 3′ end of the env gene and the polypurine tract) (Palu, G., Parolin et al., C., Rev Med Virol. 2000 10 185-202).

The majority of retroviral vectors are based on Moloney murine leukemia virus (Mo-MLV) and contain a packaging signal extending to the 5′ coding region of the gag gene (ψ⁺) with a replacement of the ATG initiation codon of the gag gene into TAG termination codon. It is generally believed that a sequence element necessary for an efficient nuclear-cytoplasmic transport of RNA molecules is located within the gag open reading frame (King, J. A., et al., FEBS Lett. 1998 434 367-371), and thus inclusion of this sequence in the extended packaging sequence can increase the viral titer (Armentano, D. et al., J. Virol. 1987 61 1647-1650; Bender, M. A. et al., J. Virol. 1987 61 1639-1646). In the wild type murine leukemia virus, unspliced mRNA is transported into the cytoplasm and is packaged into virion as genomic RNA, and it is also used as a template for translation of Gag-Pol fusion and Gag precursor proteins. On the other hand, Env protein is translated from a processed template RNA produced after splicing of the gag and pol coding sequences. Thus, both spliced and unspliced mRNAs are required at an appropriate proportion for a normal replication of the MLV. In the Mo-MLV-based MFG retroviral vector, a splice acceptor site obtained from the 5′ untranslated region of the env gene is introduced downstream of the extended packaging signal (Krall, W. J., et al., Gene Ther. 1996 3 37-48), and transgene proteins are translated from the spliced mRNA templates. These second-generation retroviral vectors can be produced in appropriate packaging cells with a relatively high viral titer.

It is known, however, that the extended packaging signal (ψ⁺) used in these vectors contains a CTG codon upstream of and in frame with the start codon for gag, which is frequently used to produce larger glycosylated Gag protein in the wild type viruses (Edwards, S. A. and Fan, H., J. Virol. 1979 30 551-563). This CTG codon can also be used in the recombinant virus to produce truncated viral protein with a potential immunogenic problem. In order to prevent this problem and to increase viral titer, Miller and co-workers developed MoMSV (Moloney murine sarcoma virus) and MoMLV hybrid vectors (collectively termed as LN series vectors) by replacing the upstream region of the MoMLV vector including sequences starting from the 5′ LTR down to the TAG termination codon introduced to replace the gag gene initiation codon with an equivalent region of the MoMSV (Miller, A. D. and Rosman, G. J., Biotechniques. 1989 7 980-982, 984-986, 989-990). The sequence of MoMSV is highly homologous to MoMLV sequence but does not produce the glycosylated Gag protein.

Although these improved vectors are widely used in a variety of applications, all of these vectors contain residual gag and/or pol coding sequences in the ψ⁺ and the splice acceptor sites, respectively. These residual sequences can be used for the generation of replication competent retroviruses (RCR) via recombination with the homologous sequences of the gag and pol genes introduced in the packaging system. It is possible that such RCR pose safety concerns especially during clinical trials. Thus, there is a need in the art to develop vectors that circumvent this potential safety concern.

The development of self-inactive (SIN) retroviral vectors was introduced as an RCR preventative measure. SIN vectors are designed so that a portion of the 3′ LTR, usually the enhancer or promoter sequences in the U3 region, has been deleted in the retroviral genome. This deletion is carried upon reverse transcription to the proviral DNA. Any transcriptional activity guided by the LTR will be altered as a result, and the absence of full length RNA results in inactive proviruses.

SIN vectors, despite their increase in degree of safety, have proven to have neither the efficiency of infection nor the level of expression needed for a successful gene therapy vector or has failed to match the degree of efficiency found in other, previously designed vectors. The present application discloses a SIN vector that is both highly infective, and demonstrates sustained high levels of expression.

The invention is directed to a retroviral SIN vector which is highly infective and demonstrates a sustained high level of expression. Typically, other SIN vectors show expression levels ranging from 10 to 100 fold lower than regular retroviral constructs. Significantly, the inventive SIN vector showed the same level of expression as the control non-SIN constructs and a popularly used SIN vector pQCXIN. In addition, extended packaging sequences including the front part of gag gene was removed in the inventive SIN vector pCS2 to increase the safety further. Data suggest that by adding the appropriate elements of safety to the construct, efficiency of infection and expression is not compromised.

Thus, the inventive vector successfully incorporates the characteristics needed for a highly effective and highly efficient gene therapy vector while maintaining the safety factors provided by self-inactivating elements.

### SUMMARY OF THE INVENTION

The present invention is directed to a SIN vector.

In one aspect, the invention is directed to a viral vector comprising the following elements, preferably in the 5′ to 3′ direction: (1) a promoter in U3 region of MSV 5′LTR; (2) repeating unit of MSV 5′LTR; (3) U5 region of MSV 5′LTR; (4) packaging signal; (5) a promoter; (6) internal ribosome entry site (IRES); (7) defective U3 region of MLV 3′ LTR; (8) repeating unit of MLV 3′ LTR; and (9) U5 region of MLV 3′ LTR.

It is understood that by a defective U3 region of MLV 3′ LTR, it is meant to indicate mutated region as well as partial or full deletions of the region so as to result in the self inactivating functionality of the vector.

The promoter used in the inventive vector may be a eukaryotic promoter, or a eukaryotic viral promoter, and in particular CMV promoter. Further, the IRES segment may be derived from any source, preferably a viral source, including but not limited to ECMV.

The vector may further include an exogenous gene such as a cytokine or any other gene. Preferably the gene may be useful in gene therapy.

In another aspect, the invention is directed to a host cell comprising the vector described above.

In another aspect, the invention is directed to a method of expressing an exogenous gene in a host mammal comprising inserting the above-described vector to a mammal in need thereof.

In yet another aspect, the invention is directed to a method of expressing an exogenous gene in a host mammal comprising transducing a mammalian cell with the above-described vector, and transplanting the mammalian cell into the mammal in need thereof.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows construction scheme of pCS2 vector. pXS was constructed by replacing the SV40/Neo/LTR of pCXSN-1 (an intermediate vector construction as discussed in Example 7 of U.S. Patent Application Publication No. 2006/0019396, published Jan. 26, 2006, the contents of which are incorporated by reference in their entirety) with the BamH1/Stu1 region (IRES/Neo/LTR region (2.8 kb fragment)) of pQCXIN (BD Biosciences, San Hose, Calif.). This region contains the Internal Ribosomal Entry Site (IRES), the Neomycin marker (Neo), and a 3′LTR with a deletion of the U3 region. This deletion duplicates to the 5′ LTR when it integrates into the chromosome and the 5′ LTR promoter is inactivated. pXS became the backbone for pCS2. To create pCS2, the PCMV IE of pVSVG (1.3 kb fragment of XbaI/XhoI digestion) was inserted into the BamHI/EcoRI cloning site using two linkers (EZClone Systems): BamHI/XbaI and XhoI/EcoRI.

FIG. 2 shows a schematic diagram of various retroviral vectors. MFG is a MoMLV-based vector and contains an extended packaging signal (ψ⁺) and SA site from the env gene 5′ untranslated region. It also contains 3′ end of env coding sequence upstream of the 3′ LTR. LN Vector is a MoMSV/MoMLV hybrid-based vector, and contains 5′ LTR and the packaging sequence obtained from MoMSV and extended packaging signal extending to gag coding region. pQCXIN vector is a LN-based vector, but is a self-inactivating (SIN) vector as it contains a deletion in the U3 region of the 3′ LTR. Instead, an internal CMV promoter is used for the expression of the transgene. pQCXIN contains an extended packaging signal (ψ⁺) and SA site from the env gene 5′ untranslated region. An SA site taken from an intron/exon junction of either the chimpanzee EF1-α gene (for pSe-BMP2) or the human CMV MIEP gene (for pScFIN) replaces the extended region of the packaging signal. It also contains 3′ end of env coding sequence upstream of the 3′ LTR. Extended packaging signal and SA site was removed in pCS2 vector. Due to the deletion of U3 region of 3′LTR, CMV promoter and intervening sequences were added as internal promoter in front of the IRES and neomycin resistance gene. Luciferase gene and BMP2 gene were introduced in front of the IRES of pCS2 as a reporter gene respectively.

FIG. 3 shows titers of retroviral vectors. The MOI of SIN vectors (pCS2, pCS2BMP2, and pCS2Luc) are similar to a control non-SIN regular vector pScFIN. The inventive SIN vectors yield 3×10⁶ cfu/ml on average.

FIGS. 4A and 4B show luciferase activities in packaging (GP2-293) and target cells (NIH3T3). FIG. 4A shows transgene (luciferase) expression in the packaging cells, in which the level of transgene expression of the self-inactivating (SIN) vector pCS2-luc was significantly higher than that of the regular vector pScFIN. FIG. 4B shows efficiency of transduction in transiently transduced NIH 3T3 cells in which the level of reporter gene expression (luciferase) in NIH 3T3 target cells was measured approximately 48 hrs after transduction.

FIGS. 5A and 5B show stable expression of BMP2 in target cells. Level of luciferase from the single clones was measured for the purpose of studying the efficiency of transgene expression from the incorporated retroviral vectors. FIG. 5A shows BMP2 activities of single clones in HDF (Human Dermal Fibroblast) cell. FIG. 5B shows BMP2 activities of single clones in HOb (Human Osteoblast) cell.

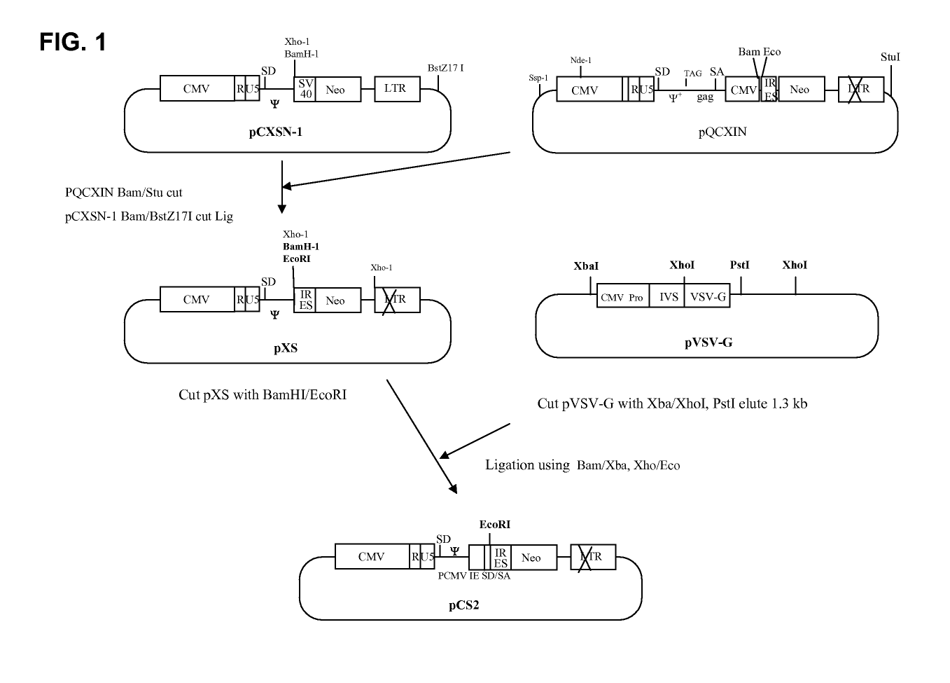

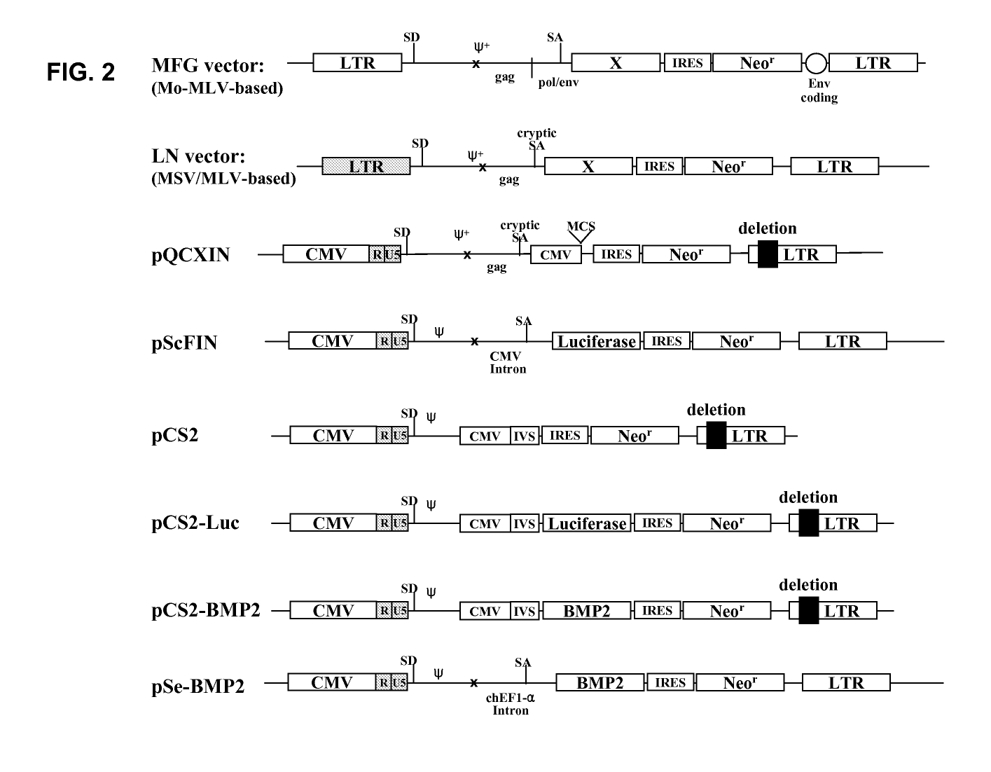

### DETAILED DESCRIPTION OF THE INVENTION

In the present application, “a” and “an” are used to refer to both single and a plurality of objects.

Inventive SIN Vector Functionality

In U. S. Patent Application Publication No. 2006/0019396, published Jan. 26, 2006, a MoMSV/MoMLV hybrid vector with an enhanced transcriptional efficiency is described, the contents relating to this subject matter being incorporated herein by reference. The possibility of RCR production is lowered significantly since the possibility of recombination between the vector and the retroviral sequences in the packaging cell is greatly reduced due to the removal of gag, pol and env genes in the vector.

In one embodiment of the invention, the present patent application describes a vector that further improves upon the vectors described in U.S. Patent Application Publication No. 2006/0019396 by adding the SIN feature to it.

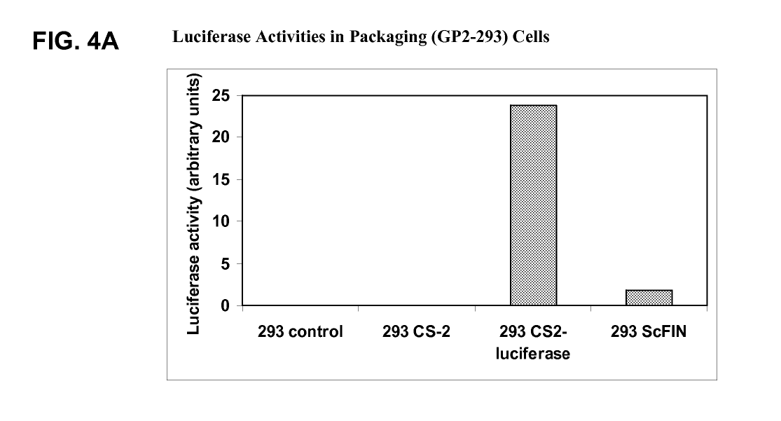

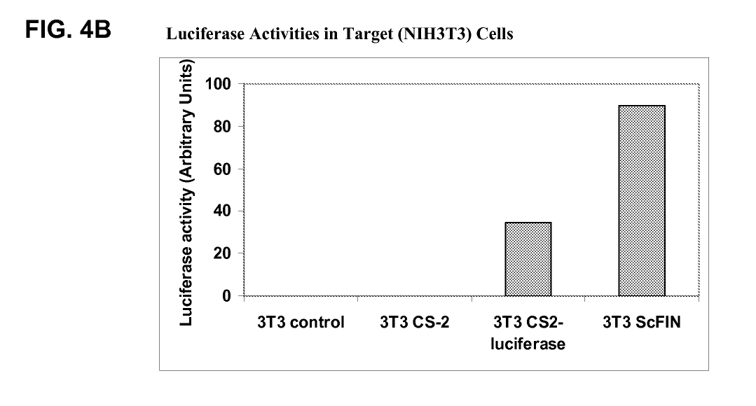

The efficiency of structural RNA generation to be packaged into viral particles was indirectly estimated by measuring the level of reporter gene expression from the GP2-293 packaging cells co-transfected with retroviral vectors and VSVG DNA. The level of transgene expression of the self-inactivating (SIN) vector pCS2-luc was significantly higher than that of the regular vector pScFIN (FIG. 4A). It is probably due to the two promoters which is located in the 5′ LTR and the internal CMV promoter in the front of the transgene.

The inventive hybrid-based retroviral SIN vector (pCS2) showed multiplicity of infection (MOI) about 3×10⁶ which is about same as commercially available vector. The inventive vector also showed constant expression of the transgene BMP-2 in NIH3T3, human dermal fibroblast, and human osteoblast cells. This vector design successfully incorporates the characteristics needed for highly effective and highly efficient gene therapy vector while maintaining the safety factors provided by self-inactivating elements.

Transforming Growth Factor-β (TGF-β) Superfamily

Transforming growth factor-β (TGF-β) superfamily encompasses a group of structurally related proteins, which affect a wide range of differentiation processes during embryonic development. This is based on primary amino acid sequence homologies including absolute conservation of seven cysteine residues. The family includes, Müllerian inhibiting substance (MIS), which is required for normal male sex development (Behringer, et al., Nature, 345:167, 1990), Drosophila decapentaplegic (DPP) gene product, which is required for dorsal-ventral axis formation and morphogenesis of the imaginal disks (Padgett, et al., Nature, 325:81-84, 1987), the Xenopus Vg-1 gene product, which localizes to the vegetal pole of eggs (Weeks, et al., Cell, 51:861-867, 1987), the activins (Mason, et al., Biochem, Biophys. Res. Commun., 135:957-964, 1986), which can induce the formation of mesoderm and anterior structures in Xenopus embryos (Thomsen, et al., Cell, 63:485, 1990), and the bone morphogenetic proteins (BMP's, such as BMP-2 to BMP-15) which can induce de novo cartilage and bone formation (Sampath, et al., J. Biol. Chem., 265:13198, 1990). The TGF-β gene products can influence a variety of differentiation processes, including adipogenesis, myogenesis, chondrogenesis, hematopoiesis, and epithelial cell differentiation (for a review, see Massague, Cell 49:437, 1987), which is incorporated herein by reference in its entirety.

The proteins of the TGF-β family are initially synthesized as a large precursor protein, which subsequently undergoes proteolytic cleavage at a cluster of basic residues approximately 110-140 amino acids from the C-terminus. The C-terminal regions of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. For most of the family members that have been studied, the homodimeric species has been found to be biologically active, but for other family members, like the inhibins (Ung, et al., Nature, 321:779, 1986) and the TGF-β's (Cheifetz, et al., Cell, 48:409, 1987), heterodimers have also been detected, and these appear to have different biological properties than the respective homodimers.

Members of the superfamily of TGF-β genes include TGF-β3, TGF-β2, TGF-β4 (chicken), TGF-β1, TGF-β5 (Xenopus), BMP-2, BMP-4, Drosophila DPP, BMP-5, BMP-6, Vgr1, OP-1/BMP-7, Drosophila 60A, GDF-1, Xenopus Vgf, BMP-3, Inhibin-βA, Inhibin-βB, Inhibin-α, and MIS. These genes are discussed in Massague, Ann. Rev. Biochem. 67:753-791, 1998, which is incorporated herein by reference in its entirety.

Bone Morphogenetic Protein (BMP)

BMPs are proteins which act to induce the differentiation of mesenchymal-type cells into chondrocytes and osteoblasts before initiating bone formation. They promote the differentiation of cartilage- and bone-forming cells near sites of fractures but also at ectopic locations. Some of the proteins induce the synthesis of alkaline phosphatase and collagen in osteoblasts. Some BMPs act directly on osteoblasts and promote their maturation while at the same time suppressing myogenous differentiation. Other BMPs promote the conversion of typical fibroblasts into chondrocytes and are capable also of inducing the expression of an osteoblast phenotype in non-osteogenic cell types. The BMP family belonging to the TGF-β superfamily comprises:

BMP-2A or BMP-2-α (114 amino acids) has been renamed BMP-2. Human, mouse and rat proteins are identical in their amino acid sequences. The protein shows 68 percent homology with Drosophila.

BMP-2B or BMP-2-β (116 amino acids) has been renamed BMP-4. Mouse and rat proteins are identical in their protein sequences.

BMP-3 (110 amino acids) is a glycoprotein and is identical to Osteogenin. Human and rat mature proteins are 98 percent identical.

BMP-3b (110 amino acids) is related to BMP-3 (82 percent identity). Human and mouse proteins show 97 percent identity (3 different amino acids). Human and rat protein sequences differ by two amino acids. The factor is identical with GDF-10.

BMP-4 is identical with BMP-2B and with DVR-4. The protein shows 72 percent homology with Drosophila.

BMP-5 (138 amino acids). At the amino acid level human and mouse proteins are 96 percent identical.

BMP-6 (139 amino acids) is identical with DVR-6 and vegetal-specific-related-1.

BMP-7 (139 amino acids) is identical with OP-1 (osteogenic protein-1). Mouse and human proteins are 98 percent identical. The mature forms of BMP-5, BMP-6, and BMP-7 show 75 percent identity.

BMP-8 (139 amino acids) is identical with OP-2. The factor is referred to also as BMP-8a.

BMP-8b (139 amino acids) is identical with OP-3 and has been found in mice only. The factor is known also as OP-3.

BMP-9 (110 amino acids) is also referred to as GDF-5.

BMP-10 (108 amino acids) has been isolated from bovine sources. Bovine and human proteins are identical.

BMP-11 (109 amino acids) has been isolated from bovine sources. Human and bovine sequences are identical. The protein is referred to also as GDF-11.

BMP-12 (104 amino acids) is known also as GDF-7 or CDMP-3.

BMP-13 (120 amino acids) is the same as GDF-6 and CDMP-2.

BMP-14 (120 amino acids) is the same as GDF-5 and CDMP-1.

BMP-15 (125 amino acids) is expressed specifically in the oocyte. The murine protein is most closely related to murine GDF-9.

Some of these proteins exist as heterodimers. OP-1, for example, associates with BMP-2A.

Because of the high degree of amino acid sequence homology (approximately 90 percent), BMP-5, BMP-6, and BMP-7 are recognized as a distinct subfamily of the BMPs. The genes encoding BMP-5 and BMP-6 map to human chromosome 6. The gene encoding BMP-7 maps to human chromosome 20. BMPs can be isolated from demineralized bones and osteosarcoma cells. They have been shown also to be expressed in a variety of epithelial and mesenchymal tissues in the embryo. Some BMPs (for example, BMP-2 and BMP-4) have been shown to elicit qualitatively identical effects (cartilage and bone formation) and to have the ability to substitute for one another.

Gene Therapy

In a specific embodiment, nucleic acids comprising sequences encoding any therapeutic polypeptide are included in the inventive vector and are administered to treat, inhibit or prevent a disease or disorder associated with aberrant expression and/or activity of the polypeptide, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the vector nucleic acid sequences may contain a therapeutic polypeptide expressible in a suitable host. In particular, such nucleic acid sequences have promoters operably linked to the polypeptide coding region, said promoter being inducible or constitutive, and, optionally, tissue-specific. In another particular embodiment, nucleic acid molecules are used in which the polypeptide coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989).

Delivery of the nucleic acids into a patient may be either direct, in which case the patient is directly exposed to the nucleic acid or nucleic acid- carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retroviral or other viral vectors.

In one embodiment, the nucleic acid is introduced into a cell prior to administration in vivo of the resulting recombinant cell. Such introduction can be carried out by any method known in the art, including but not limited to transfection, electroporation, microinjection, infection with the inventive vector containing the nucleic acid sequences, cell fusion, chromosome-mediated gene transfer, microcell-mediated gene transfer, spheroplast fusion and so on. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and so on.

In a preferred embodiment, the cell used for gene therapy is allogeneic to the patient, although autologous cells may be used as well.

In an embodiment in which recombinant cells are used in gene therapy, nucleic acid sequences encoding the polypeptide are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Therapeutic Composition

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, i.e., the brain, thus requiring only a fraction of the systemic dose.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

### Example 1.1

### Vector Construction

pXS was constructed by replacing the SV40/Neo/LTR of pCXSN-1 (an intermediate vector construction as discussed in Example 7 of U.S. Patent Application Publication No. 2006/0019396, published Jan. 26, 2006, the contents of which are incorporated by reference in their entirety) with the BamH1/Stu1 region of pQCXIN (BD Biosciences, San Hose, Calif.). This region contains the Internal Ribosomal Entry Site (IRES), the Neomycin marker (Neo), and a 3′ LTR with a deletion of the U3 region. This deletion duplicates to the 5′ LTR when it integrates into the chromosome and the 5′ LTR promoter is inactivated. pXS became the backbone for pCS2. To create pCS2, the PCMV IE of pVSVG (1.3 kb fragment of XbaI/XhoI digestion) was inserted into the BamHI/EcoRI cloning site using two linkers (EZClone Systems): BamHI/XbaI and XhoI/EcoRI.

Luciferase gene was used as a reporter gene. The 1.6 kb gene from an EcoRI digestion of pDEFL-1 was cloned into pCS2. The BMP2 gene was also used as a reporter gene. pMTBMP2 was digested with EcoRI to obtain the insert and inserted the fragment containing BMP2 into pCS2. The vectors for Luciferase and BMP2 were named pCS2-Luc and pCS2-BMP2, respectively.

pCXSN is the first evolutionary step in the construction of the vector, contained the extended hCMV enhancer/promoter region from pQCXIN and the U3 sequence from the 5′ LTR of pLXSN.

pCXSN-1 was generated after the removal of the extended packaging signal with the 5′ coding region of the gag gene. Splicing acceptor signal was also removed. Minimum length of packaging signal provide increased safety by reducing the chances of recombination in a packaging cell line.

### Example 1.2

### Production of Retroviral Supernatants

VSV-G pseudo-typed vector particles were produced by transiently co-transfecting GP2-293 cells with a retroviral vector DNA and VSV-G plasmid, following the method previously described in U.S. Patent Application Publication No. 2006/0019396, published Jan. 26, 2006. The material relating to this subject matter is hereby incorporated by reference herein.

### Example 1.3

### Transduction to Target Cells

VSV-G pseudo-typed vector particles were produced by transiently co-transfecting GP2-293 cells with a retroviral vector and VSV-G plasmid, following the method previously described in U.S. Patent Application Publication No. 2006/0019396, published Jan. 26, 2006. The material relating to this subject matter is hereby incorporated by reference herein. 293 cells were maintained in Dulbecco's modified medium without phenol red and supplemented with 10% Fetal Bovine serum. Cells were transfected on collagen-coated 6 well plates at 1×10⁶ cells per well, using 12 ul of Fugene-6 (Roche) and 2 ug of plasmid per well with 2 ml of medium. Cell medium was collected and filtered for transduction 2 days after transfection. The process was repeated the next day. To measure the stable transduction efficiency, transduced cells were selected for neomycin resistance using G-418.

### Example 1.4

### Titering Retroviral Vectors

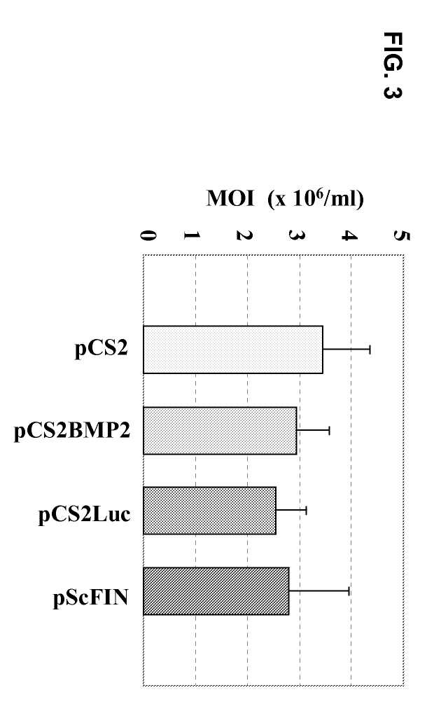

To obtain the titration of retroviral vectors, NIH 3T3 cells were grown in 6 well plates as described above and transduced with virus containing supernatant obtained from GP2-293 cells 48 hr after transfection with retroviral vectors in serial dilutions. To concentrate the viral particles, GP2-293 cell transduced with a retroviral vector was grown in 10 cm dishes in 6 ml of D-10 medium. Viral supernatants obtained from two 10 cm GP2-293 cell dishes were pooled together, filtered and centrifuged at 50,000×g in an SW41 ultracentrifuge rotor at 4° C. for 90 min. Virus pellets were resuspended in 30 μl of N-10 medium by shaking at room temperature for 90 min. Ultracentrifuged viruses were diluted 100 times in the same medium before using in the titering experiment. Approximately 36 hrs after transduction with serially diluted viral supernatants, cells were replaced with G-418 containing medium at increasing concentrations between 0.3 mg/ml and 1 mg/ml, and allowed to grow for an additional 12-14 days until distinct G-418-resistant colonies are formed, replacing medium from cells every two days. G-418 resistant colonies were counted (FIG. 3).

### Example 1.5

### Determination of Transcriptional Efficiency from Transduced G-418-Resistant Single Clones

For the determination of transcriptional efficiency from NIH 3T3 cells transduced with retroviral vectors, 5-6 single clones of transduced cells were picked using 5 mm diameter sterile cloning disks (Sigma-Aldrich Corp, St. Louis, Mo.) according to the manufacturer's protocol. Single colonies were picked from NIH 3T3 plates used for titering retroviral vectors 12-14 days after the start of G-418 selection, from wells inoculated with retroviral supernatant at the highest possible dilution. Colonies picked using the disk were transferred to 12 well plates and allowed to grow for 4 days, and split into fresh 12 well plates at roughly equal densities estimated based on the amount of growth after 4 days. For luciferase producing cells, luciferase assay were performed. For BMP2 producing cells, BMP2 ELISA assay kits (R&D Systems, MN) were used to determine their expression in the transduced cells.

### Example 1.6

### Luciferase Reporter Assay

Cells were first trypsinzed and counted for cell numbers, collected by centrifugation at 1,000 rpm for 4 min, and lysed using 0.5 ml of RLB for the luciferase assay. Samples were stored at −80° C. until ready for the assay.

Samples were thawed before the assay, vortexed 8 times for 1 sec, and centrifuged at 12,000 rpm in a micro-centrifuge for 15 sec to remove cell debris. The luciferase assay was performed using either 10 or 20 μl of cell lysates (after appropriate dilutions in the RLB as indicated in the figure legends) by adding 100 μl of the assay buffer containing the substrate for the enzyme in 96 well plates, and luciferase activity was measured for 10 seconds after 2 second delay using the LB960 luminometer (Berthold Technologies, Oak Ridge, Tenn.).

### Example 1.7

### Detection of Replication Competent Retroviruses from Viral Supernatant

An RCR test was performed following an extended S⁺/L⁻ assay (reported in Chen et al., Hum. Gen. Ther. 2001, 12:61-70, the contents of which are incorporated by reference in its entirty), which includes 3-week amplification of virus on the permissive Mus dunni cell line and detection of RCR on the feline PG-4 cell line by the formation of transformed foci when RCR is present. Both M. dunni cells and PG-4 cells were maintained in McCoy's 5A modified medium supplemented with 10% fetal bovine serum (M-10). M. dunni cells were seeded in T25 flasks at 2×10⁵ per flask in 6 ml of M-10 medium one day before transduction, and changed with 3 ml of fresh medium containing 16 μl/ml of polybrene 1 hr before transduction. Three (3) ml of filtered viral supernatant collected from 3 wells of GP2-293 cells transfected with each retroviral vector for 48 hrs in 6 well plates was then added to the M. dunni cell flask. Cells were allowed to grow for 3 weeks; passaging two times per week. After the final passage, cells were allowed to grow an additional 2-3 days to become confluent, replaced with fresh medium, and allowed to grow for 1 more day. The supernatant from each flask was collected, filtered through 0.45 μm syringe filter and used in the focus-forming assay. PG-4 cells were seeded in 6 well plates at 1×10⁵ cells per well one day before the assay, and re-fed with 1 ml of medium containing 16 μg/ml of polybrene just prior to the inoculation. One ml of filtered supernatant from M. dunni cells was then added to PG-4 cells (in duplicate) and the formation of discernible foci was checked under the microscope 4-5 days after the inoculation.

### Example 2

### Results

### Example 2.1

### Titer Determination of the Virus

This method relies on the efficiency of the expression of the neomycin resistance gene driven by the viral LTR promoter (pScFIN) or by the internal CMV promoter (pCS2BMP2 and pCS2Luc) through the use of IRES. Based on this method, the inventive SIN vectors achieved about 3×10⁶ cfu/ml (FIG. 3). As these vectors are pseudotyped with VSV-G proteins, we tried to concentrate and increase the titer of virus by ultracentrifugation. One time ultracentrifugation of the viral supernatant increased viral titer 50-100 fold over the original titer, recording up to 5×10⁸ cfu/ml.

### Example 2.2

### Transgene Expression in the Packaging Cell

The efficiency of structural RNA generation to be packaged into viral particles was indirectly estimated by measuring the level of reporter gene expression from the GP2-293 packaging cells co-transfected with retroviral vectors and VSVG DNA. The level of transgene expression of the self-inactivating (SIN) vector pCS2-luc was significantly higher than that of the regular vector pScFIN (FIG. 4A).

### Example 2.3

### Efficiency of Transduction in Transiently Transduced NIH 3T3 Cells

The efficiency of transduction by various retroviral vectors was indirectly estimated by measuring the level of reporter gene expression in NIH 3T3 target cells approximately 48 hrs after transduction. The efficiency of transient transduction of pCS2-Luc vector was 40-50% lower than that of the vector pScFIN (FIG. 4B).

### Example 2.4

### Efficiency of Expression from Stably Transduced Single Clones

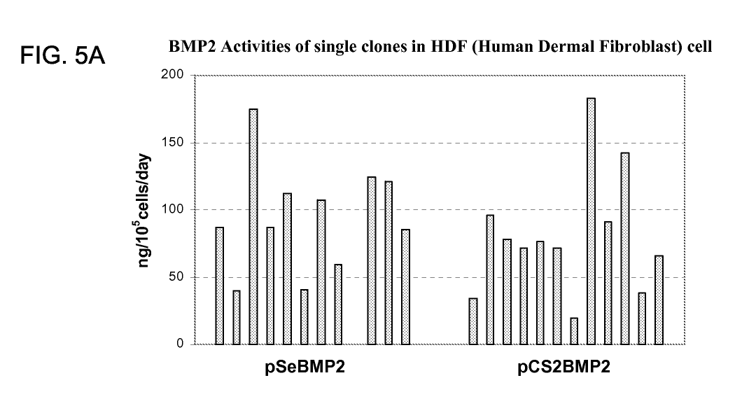

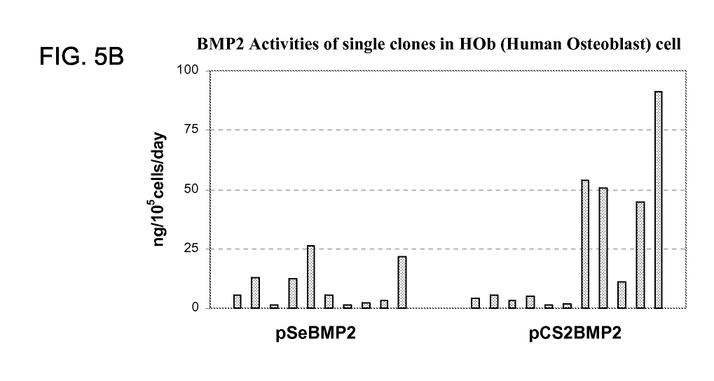

To study the efficiency of transgene expression from the incorporated retroviral vectors, the levels of luciferase from the single clones was measured. To minimize the effects of multiple incorporations, G418-resistant single colonies were picked from the 6 well plates in wells transduced with the highest possible dilution of the viral supernatant. Therefore, the levels of reporter gene expression from these single cell clones transduced with various vectors can be directly compared for the efficiency of transcription after a single stable integration into the host genome. FIG. 5 shows that the efficiencies of transcription were variable among clones, reflecting the positional effects anticipated due to the random incorporation of retroviral vectors within the host genome. The efficiency of transcription from pCS2BMP2 which is a SIN vector showed similar efficiency with a regular vector pSeBMP2. An increase in transcriptional efficiency after retroviral incorporation allows for the use of the viral supernatant at a significantly lower viral titer for the transduction of target cells, and reduces the chance for the incorporation of the virus at an undesirable location, and makes the pre-screening procedure easier. Furthermore, removal of gag, pol, and env genes facilitates RCR-free retroviral gene transfer while enabling improved efficacy.

### Example 2.5

### No RCR Production

No RCR was detected in any of the retroviral vector preparations (No data shown).

### Example 3

### Transduction Efficiency

Transduction efficiency of three (3) forms of pCS2 was checked: pCS2, which is an empty vector with no included transgene; pCS2-luciferase, which is pCS2 in which luciferase gene is inserted as a transgene; and pCS2BMP2, which is BMP2 gene is inserted into pCS2 vector as a transgene.

In the past, low transduction efficiency of SIN vector was one of the factors that caused the SIN vector to be inferior compared with conventional retroviral vectors. Table 1 shows the transduction efficiency of the various indicated vectors. The inventive SIN vector as exemplified by pCS2 showed similar MOI (multiplicity of infection) to a convectional vector such as pSeBMP2. The inventive SIN vector also showed consistent MOI compared with commercial SIN vector CFIN-CM.

TABLE 1
Transduction Efficiency of Various Vectors

| Data Type | Volume of | | | |
|---|---|---|---|---|
| DNA | Supernatant | Colony # | Dilution | MOI |
| pCS2 | | | | |
| 1 | 0.5 | 13 | 1.00 × 10⁺⁰² | 2.60 × 10⁺⁰³ |
| 2 | 0.5 | 6 | 1.00 × 10⁺⁰⁵ | 1.20 × 10⁺⁰⁶ |
| 3 | 0.5 | 24 | 1.00 × 10⁺⁰⁴ | 4.80 × 10⁺⁰⁵ |
| 4 | 0.5 | 28 | 1.00 × 10⁺⁰⁶ | 5.60 × 10⁺⁰⁷ |
| 5 | 0.5 | 8 | 1.00 × 10⁺⁰⁶ | 1.60 × 10⁺⁰⁷ |
| 6 | 0.5 | 9 | 1.00 × 10⁺⁰⁶ | 1.80 × 10⁺⁰⁷ |
| 7 | 0.5 | 19 | 1.00 × 10⁺⁰⁶ | 3.80 × 10⁺⁰⁷ |
| 8 | 0.5 | 14 | 1.00 × 10⁺⁰⁶ | 2.80 × 10⁺⁰⁷ |
| Average | | | | 1.97 × 10⁺⁰⁷ |
| pCS2- | | | | |
| luciferase | | | | |
| 1 | 0.5 | 15 | 1.00 × 10⁺⁰⁴ | 3.00 × 10⁺⁰⁵ |
| 2 | 0.5 | 13 | 1.00 × 10⁺⁰⁶ | 2.60 × 10⁺⁰⁷ |
| 3 | 0.5 | 17 | 1.00 × 10⁺⁰² | 3.40 × 10⁺⁰³ |
| 4 | 0.5 | 2 | 1.00 × 10⁺⁰³ | 4.00 × 10⁺⁰³ |
| 5 | 0.5 | 8 | 1.00 × 10⁺⁰⁶ | 1.60 × 10⁺⁰⁷ |
| 6 | 0.5 | 13 | 1.00 × 10⁺⁰⁶ | 2.60 × 10⁺⁰⁷ |
| 7 | 0.5 | 16 | 1.00 × 10⁺⁰⁶ | 3.20 × 10⁺⁰⁷ |
| 8 | 0.5 | 14 | 1.00 × 10⁺⁰⁶ | 2.80 × 10⁺⁰⁷ |
| Average | | | | 1.60 × 10⁺⁰⁷ |
| pCS2BMP2 | | | | |
| 1 | 0.5 | 10 | 1.00 × 10⁺⁰⁶ | 2.00 × 10⁺⁰⁷ |
| 2 | 0.5 | 14 | 1.00 × 10⁺⁰⁶ | 2.80 × 10⁺⁰⁷ |
| 3 | 0.5 | 19 | 1.00 × 10⁺⁰⁶ | 3.80 × 10⁺⁰⁷ |
| 4 | 0.5 | 2 | 1.00 × 10⁺⁰⁶ | 4.00 × 10⁺⁰⁶ |
| 5 | 0.5 | 13 | 1.00 × 10⁺⁰⁶ | 2.60 × 10⁺⁰⁷ |
| 6 | 0.5 | 14 | 1.00 × 10⁺⁰⁶ | 2.80 × 10⁺⁰⁷ |
| 7 | 0.5 | 10 | 1.00 × 10⁺⁰⁶ | 2.00 × 10⁺⁰⁷ |
| 8 | 0.5 | 17 | 1.00 × 10⁺⁰⁶ | 3.40 × 10⁺⁰⁷ |
| 9 | 0.5 | 18 | 1.00 × 10⁺⁰⁶ | 3.60 × 10⁺⁰⁷ |
| Average | | | | 2.60 × 10⁺⁰⁷ |
| pSeBMP2 | | | | |
| 1 | 0.5 | 14 | 1.00 × 10⁺⁰⁶ | 2.80 × 10⁺⁰⁷ |
| 2 | 0.5 | 10 | 1.00 × 10⁺⁰⁶ | 2.00 × 10⁺⁰⁷ |
| Average | | | | 2.40 × 10⁺⁰⁷ |
| CFIN-CM | | | | |
| 1 | 0.5 | 1 | 1.00 × 10⁺⁰¹ | 2.00 × 10⁺⁰¹ |
| 2 | 0.5 | 23 | 1.00 × 10⁺⁰⁶ | 4.60 × 10⁺⁰⁷ |
| 3 | 0.5 | 12 | 1.00 × 10⁺⁰¹ | 2.40 × 10⁺⁰² |
| 4 | 0.5 | 14 | 1.00 × 10⁺⁰⁶ | 2.80 × 10⁺⁰⁷ |
| 5 | 0.5 | 15 | 1.00 × 10⁺⁰⁶ | 3.00 × 10⁺⁰⁷ |
| 6 | 0.5 | 12 | 1.00 × 10⁺⁰⁶ | 2.40 × 10⁺⁰⁷ |
| 7 | 0.5 | 15 | 1.00 × 10⁺⁰⁶ | 3.00 × 10⁺⁰⁷ |
| 8 | 0.5 | 14 | 1.00 × 10⁺⁰⁶ | 2.80 × 10⁺⁰⁷ |
| Average | | | | 2.33 × 10⁺⁰⁷ |

Multiplicity of Infection (MOI)=1/Volume Viral Supernate×dilution×(# of Colonies).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The examples offered above are by way of illustration of the present invention, and not by way of limitation.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A viral vector comprising the following elements:
(1) a promoter in U3 region of MSV 5′LTR;
(2) repeating unit of MSV 5 ′LTR;
(3) U5 region of MSV 5′LTR;
(4) packaging signal;
(5) a promoter;
(6) internal ribosome entry site (IRES);
(7) defective U3 region of MLV 3′ LTR;
(8) repeating unit of MLV 3′ LTR; and
(9) U5 region of MLV 3′ LTR

2. The viral vector according to claim 1, wherein the promoter in element (1) is a eukaryotic promoter.

3. The viral vector according to claim 2, wherein the promoter in element (1) is a eukaryotic viral promoter.

4. The viral vector according to claim 3, wherein the promoter in element (1) is a CMV promoter.

5. The viral vector according to claim 1, wherein the promoter in element (5) is a eukaryotic promoter.

6. The viral vector according to claim 5, wherein the promoter in element (5) is a eukaryotic viral promoter.

7. The viral vector according to claim 6, wherein the promoter in element (5) is a CMV promoter.

8. The viral vector according to claim 1, wherein the IRES in element (6) is from Encephalomyocarditis virus (ECMV).

9. The viral vector according to claim 1, comprising an exogenous gene.

10. The viral vector according to claim 9, wherein the gene is a cytokine.

11. The viral vector according to claim 10, wherein the gene is a member of the TGFbeta superfamily.

12. The viral vector according to claim 11, wherein the gene is TGFbetal.

13. The viral vector according to claim 12, wherein the gene is BMP.

14. A host cell comprising the vector according to claim 1.

15. A method of expressing an exogenous gene in a host mammal comprising inserting the vector according to claim 1 to a mammal in need thereof.

16. A method of expressing an exogenous gene in a host mammal comprising transducing a mammalian cell with the vector according to claim 1, and transplanting the mammalian cell into the mammal in need thereof.
